(19) United States
(12) Patent Application Publication
(10) Pub. No.: US 2008/0319321 A1
(43) Pub. Date: Dec. 25, 2008

(54) TERAHERTZ IMAGING

(76) Inventor: Gunter Goldbach, Worth/Wifling (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 11/752,367

(22) Filed: May 23, 2007

Related U.S. Application Data
(60) Provisional application No. 60/917,089, filed on May 10, 2007.

(30) Foreign Application Priority Data
May 24, 2006 (EP) 06010634

(51) Int. Cl. A61B 6/00

(52) U.S. Cl. 600/475; 600/473

(57) ABSTRACT

A body or body structure may be examined by detecting radiation emitted from and/or reflected by the body or body structure, said radiation being in the terahertz frequency range. The detected radiation then is evaluated to obtain information concerning the body or body structure.

### RELATED APPLICATION DATA

This application claims priority of U.S. Provisional Application No. 60/917,089 filed on May 10, 2007, which is incorporated herein by reference in its entirety.

### FIELD OF THE INVENTION

The present invention relates generally to medical imaging. More particularly, the invention relates to a method and device for examining a body by means of radiation in the terahertz frequency range.

### BACKGROUND OF THE INVENTION

Various systems are known and used for medical imaging. However, such known systems have drawbacks. For example, some medical imaging systems can be damaging to the patient's health (e.g., x-ray systems), imprecise (e.g., ultrasound systems), cannot satisfactorily show soft tissue (e.g., computer tomography systems), or cannot provide a sufficiently clear image of bone material (e.g., magnetic resonance systems).

### SUMMARY OF THE INVENTION

A method for examining a patient's body, body part or body structure includes detecting radiation in the terahertz frequency range (i.e., a frequency of between 0.1 and 30 THz). The radiation may be emitted or reflected by the body, body part or body structure, wherein the radiation can be detected by a sensor or detector, for example.

Radiation emitted by the body in the terahertz frequency range can be understood to mean both the body itself emitting radiation in the terahertz frequency range as well as irradiating or transmitting through the body radiation in the terahertz frequency range. The emitted or reflected radiation of the body, body part or body structure can be detected by a terahertz sensor and evaluated or processed by a computational unit so as to obtain information concerning the body or body structure. The radiation detected by the terahertz sensor, for example, can be evaluated such that information concerning the nature, type, composition, material, shape, structure, condition, temperature or position of the surface or interior of the body or body part or body structure may be obtained.

A registration process, in particular an automatic registration process of the body, body part or body structure, for example, can be performed on the basis of the obtained information concerning the body. For example, registration may be performed by identifying landmark points on the body and assigning the landmarks to spatial positions, or by taking recordings from different known positions using a camera and further processing the recordings until the body, body part or body structure has been registered.

The information concerning the body that is ascertained by means of the terahertz radiation also can be combined with other information. Thus, for example, a plurality of recordings of the body or body part can be obtained by means of the terahertz radiation, wherein the terahertz radiation can exhibit the same frequency or frequency range or a different frequency or frequency range, such that, for example, the same body or body structures can be recorded from different positions and combined with each other, or different parts of a body, such as the surface or interior of a body, can be recorded by means of different frequencies or frequency ranges and combined with each other. Depending on the selected frequency, the radiation may slightly penetrate into the surface of the body and then may be reflected on or near the surface, or the radiation may penetrate deep into or through the body and/or penetrate through clothing. The information concerning the body, body structure or body part that is ascertained by means of the terahertz radiation also can be combined with other data by means of an image fusion process. For example, information concerning the same body, body structure or body part that may have been ascertained by another imaging method such as, for example, an x-ray method, magnetic resonance method, computer tomography method, ultrasound method, positron emission tomography (PET) method, or a single photon emission computed tomography (SPECT) method, can be combined with data ascertained by means of terahertz radiation. The information concerning the body that is ascertained from the terahertz radiation preferably is combined with two-dimensional or three-dimensional information concerning the body that has been ascertained, for example, by means of terahertz radiation or another imaging method.

The terahertz sensor, for example, can detect radiation emitted by the body part or body structure in the terahertz frequency range. The body under examination, for example, also can be irradiated with terahertz radiation, such that the radiation reflected on the body, for example on or near the surface of the body, is detected by a terahertz sensor, such as a terahertz sensor array that is arranged around the body or body part. Terahertz radiation that is irradiated or transmitted through the body also can be detected by the terahertz sensor, wherein the sensor may be arranged opposite the radiation source. Depending on the nature, type, composition, material, shape, structure, condition, temperature or position of the body or body structure or body part, a varying amount of terahertz radiation may be transmitted through the body or reflected or emitted by the body. The terahertz radiation also may be dampened or absorbed to a varying extent, such that information concerning the surface or interior of the body can be obtained from the detected terahertz radiation.

An instrument such as a microscope or endoscope, on which active or passive markers, for example, may be arranged, also can be navigated on the basis of the ascertained information concerning the body or body part. For example, it is possible to ascertain properties of the body or body part, such as the nature, shape, structure, condition or position of the body or surface of the body, by irradiating the body with terahertz radiation from a preferably known distance, and then detecting the radiation reflected on the body or surface of the body using a sensor. The shape, position or distance of the body or surface, for example, can be deduced from the transit time of the terahertz radiation or signal.

The terahertz radiation or signal detected by the sensor also can be evaluated, for example, such that the spectrum or frequency range representation of the radiation or signal may be determined by means of a Fourier transformation, and properties of the body or body part that emitted, reflected or transmitted the radiation may be deduced from the spectral properties. The ascertained spectral representation of the detected terahertz radiation, for example, can be compared with spectra of known materials, shapes, conditions or temperatures, wherein the properties of the body or body part under examination can be ascertained from the comparison.

Preferably, characteristic frequencies of the detected terahertz signal, such as frequencies of maximum or minimum absorption, reflection or transmission, can be compared with characteristic frequencies such as resonance frequencies or maximum or minimum absorption, transmission or reflection frequencies of known bodies or body structures that have been previously ascertained or stored. Based on the comparison of the frequencies, the nature, type, composition, material, shape, structure, condition, temperature or position of the body or the surface or interior of the body can be ascertained.

A body or body parts such as a head, face, arm, jaw, dentures or hand, and body structures such as a patient's tissue, bones and/or bone structures, vessels, ligaments, tendons, teeth or skin can in particular be examined by means of terahertz radiation. In a cranial application, for example, it would be possible not only to show the outer contour and/or surface of the face or head, but also to determine detailed information concerning the position of prominent bone structures beneath the skin. Tumors, for example, also can be identified on the exposed brain by detecting terahertz radiation emitted by the brain during an operation (e.g. detected by a spectral terahertz sensor) in order to obtain detailed information concerning the type of the tissue. Thus, by superimposing images in a microscope, it is possible to identify, on the basis of spectral information, what tissue is and is not tumorous. It is also possible, by means of the detected terahertz radiation, to identify tumours near the surface such as skin cancer, for example, on the basis of the different spectral characteristics of diseased and healthy tissue. In combination with a tracking system for obtaining three-dimensional information concerning the position of the tumors, a targeted treatment can be enabled, e.g., by injecting beneath the tumors or resecting the tumors. Furthermore, it is for example possible, on the basis of terahertz images, to perform a navigation process in hand surgery. Information relating to the temperature, as in the event of swelling and vascular injuries, the type and position of vessels and ligaments, for example, can be ascertained before, during or after an operation. In dental applications, the thickness of the enamel, the interior condition of the teeth or the shape of the teeth can be established, or dental caries or periodontosis can be established, by means of the terahertz radiation.

Terahertz radiation in a frequency range of between 0.1 and 5 THz is preferably used for examining the body or body structure, wherein the spectral ranges between 0.1 and 0.6 THz and between 0.5 and 2 THz are preferably used. Terahertz radiation in a range around 1.6 THz or in a range around 2.5 THz or in a wide, broadband range around 3 THz can also be used.

The method described herein may be embodied as a computer program which, when it is loaded onto a computer or is running on a computer, performs a method as described herein. The computer program may be embodied on a computer readable medium so as to form a computer program product.

A device for examining a body comprises a terahertz sensor or terahertz detector, in particular a terahertz camera, wherein the sensor can detect terahertz radiation reflected by a body or body structure, transmitted through a body or body structure, or emitted by a body or body structure.

The device also can comprise a computational unit, such as a tracking system, which may be connected to the terahertz sensor by a wired or wireless connection, thereby enabling the detected terahertz radiation or the detected data to be transmitted to the computational unit for evaluation and/or further processing. The detected terahertz radiation can be evaluated in the computational unit in order to obtain information concerning the body or body structure. Preferably, the terahertz signal detected by means of the terahertz sensor can be analyzed, such as for example transformed into the frequency range by means of a Fourier transformation, such that characteristic frequencies such as absorption frequencies or resonance frequencies can be ascertained from the spectrum of the detected terahertz signal.

The device also can comprise a terahertz radiation source, in particular terahertz lamps, which can emit terahertz radiation for irradiating the body or body structure. The terahertz radiation source can be connected to the terahertz sensor or to the computational unit. The radiation emitted by the terahertz radiation source, for example, can be reflected on the body structure or body part to be examined or can be transmitted through the body or body structure, such that the reflected or transmitted radiation can be detected by the terahertz sensor. The terahertz radiation source can be arranged at a known location or can be provided with markers such that, for example, the position of the terahertz radiation source can be ascertained by a tracking system.

If, for example, a transmission measurement of a body is taken, then the terahertz radiation source is preferably situated opposite the terahertz sensor, such that radiation emitted by the terahertz radiation source at least partly penetrates through the body or body structure and can be detected on the opposite side by the terahertz sensor. If, for example, a reflection measurement is taken, then the terahertz radiation source can be arranged at a location that is known or can be ascertained, wherein the terahertz sensor can be arranged, for example as a sensor array, around the body or body structure so as to detect the terahertz radiation that is at least partially reflected from the body.

The device also can comprise a database that may be connected to the computational unit, wherein the database includes characteristic information on a plurality of bodies or body structures. Characteristic frequencies, such as absorption frequencies or resonance frequencies, of particular body parts or body structures, such as tissue, bones, vessels, ligaments, tendons, teeth or skin, can be stored in the database. Further, characteristic spectra of a plurality of body parts or body structures, which can serve as a spectral fingerprint of a body or body structure, can be stored in the database. Preferably, the computational unit can compare the information concerning the body or body structure as obtained by means of the detected terahertz radiation with the information stored in the database, and in particular compare the characteristic frequencies or the spectrum and/or frequency range, and draw conclusions from the comparison regarding the nature, type, composition, material, shape, structure, condition, temperature or position of the surface or interior of the body. Characteristic frequencies of the detected terahertz signal or the spectrum of the detected terahertz signal may be compared with the stored information, and the presence of a particular material or a particular structure or temperature of the body, for example, can be deduced from the similarity in characteristic frequencies or spectra.

The device also can comprise a data output device, in particular a screen, which can display the ascertained information concerning the body as numerical values or as an image, or can show fused or registered body structures. In addition, a data input device can be connected to the computational unit or database, in particular a keyboard or a scanner such as an x-ray device, a computer tomograph, a nuclear spin tomograph, an ultrasound tomograph, a positron emission tomograph or a single photon emission tomography tomograph. Information can be input into the computational unit or database by means of the data input device, such that the information can be stored in the database or can be compared, for example, with the information obtained by means of the detected terahertz radiation, or processed in the computational unit.

The terahertz radiation source or terahertz sensor preferably comprises an electronic or optical terahertz oscillator, such as a titanium-sapphire laser.

### BRIEF DESCRIPTION OF THE DRAWINGS

The forgoing and other features of the invention are hereinafter discussed with reference to the drawings.

FIG. 1 is a schematic diagram of an exemplary system for examining a body using terahertz radiation in accordance with the invention.

FIG. 2 is an exemplary graph of detected terahertz radiation in the time domain.

### DETAILED DESCRIPTION

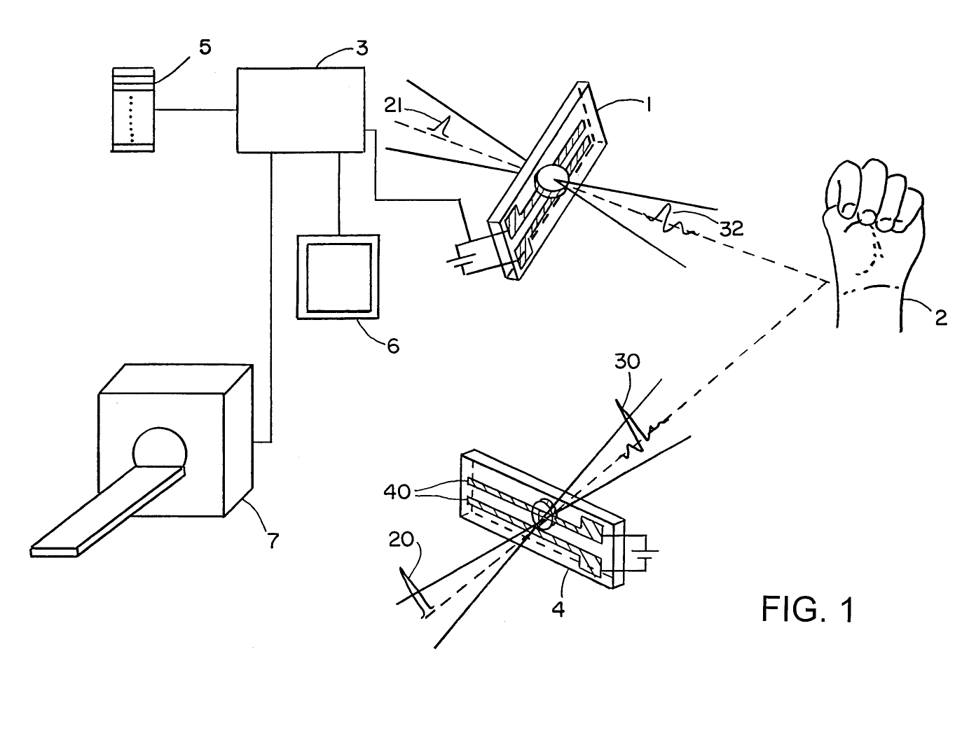

FIG. 1 shows an exemplary system for examining a body in accordance with the invention, wherein a body or body part, such as a hand 2, is irradiated by a terahertz radiation source 4. The terahertz radiation source 4 preferably comprises a mode-coupled titanium-sapphire laser that can emit pulses 20 having a duration of several femtoseconds (e.g., 10-50 femtoseconds). These optical pulses 20 can be indexed to a photoconductive dipole antennae 40, which can include gallium arsenide onto which two metal strip conduits have been metallized. The short laser pulses 20 generate charge carriers between the conduits that are accelerated by an electric field applied to the dipole antennae 40, resulting in a short pulse of current that generates a terahertz pulse 30 that may be emitted from the radiation source 4. In the present example, the terahertz pulse strikes a hand 2 and is reflected from its surface or by structures near the surface, wherein the reflected terahertz pulse 32 may be detected by a terahertz sensor 1 or terahertz detector.

The terahertz sensor 1 can have a similar design to the terahertz radiation source 4, wherein an external field need not be applied or can be configured otherwise, such as for example as a purely electronic sensor. A switching laser pulse 21 can generate free charge carriers in the terahertz sensor 1 that move in the electric field of the incoming or detected terahertz wave 32 reflected by the hand 2. This can generate a small flow of current that can be amplified and registered.

The generated current flow can be transmitted to a computational unit 3 such as a computer, where it can be further processed or evaluated. The profile over time of the detected terahertz radiation 32, for example, can be ascertained from the generated current, and the spectrum or the frequency range representation of the detected terahertz pulse 32 can be determined in the computational unit 3, for example, by means of a Fourier transformation.

The ascertained information can be output on the data output device 6, such as a screen, and compared with time domain and frequency range representations of known bodies or body structures that are stored in the database 5. In the present example, the detected terahertz pulse 32 can include information concerning the skin, surface or tissue of the hand 2, and can be compared by the computational unit 3 with information concerning known tissue that is stored in the database 5, for example. Via a data input device 7, such as a computer tomograph 7, for example, information concerning the body 2, such as the hand 2, can be detected, transmitted to the computational unit 3, and stored in the database 5 as reference data for comparison with the detected information.

Instead of the method of ascertaining the information concerning the body part by means of reflection, as shown in FIG. 1, it is also possible to use the radiation source 4 and the terahertz sensor 1 to examine the body part by transmission of terahertz radiation (i.e., transmission of terahertz radiation through the body or body part), or to simply use the terahertz sensor 1 to measure emission of terahertz radiation from the body or body part. Instead of the computer tomograph 7 shown, another input device, such as a keyboard, an x-ray device such as a C-arm, an ultrasound tomograph, a magnetic resonance tomograph, a positron emission tomograph or a SPECT tomograph may be used to obtain information concerning a body part or body 2. This information then can be evaluated in the computational unit 3 or stored as reference information in the database 5.

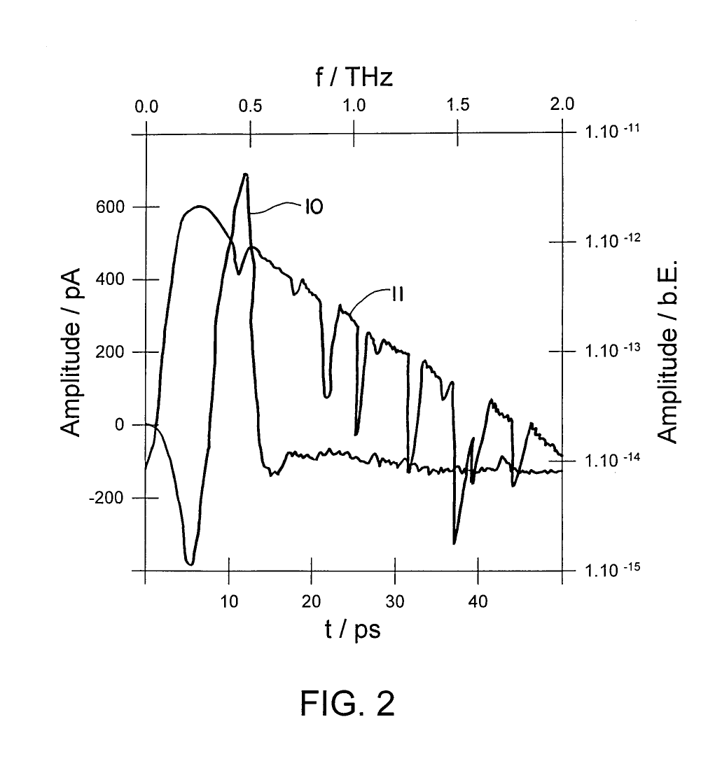

FIG. 2 shows a detected terahertz pulse in the time domain 10 and frequency range 11. The frequency range representation 11 of the detected terahertz signal can be compared with terahertz signals of known body parts or body structures such as skin or tissue, by the computational unit so as to draw conclusions regarding the body part or body structure, such as the tissue, under examination. It is possible to compare the entire frequency range representation 11 with stored frequency range representations and, by isolating the most similar frequency profiles, to deduce the type of the tissue under examination. This can enabled, for example, healthy tissue to be distinguished from diseased tissue such as skin cancer. Characteristic frequencies, such as the frequencies of maximum absorption or minimum reflection that may be seen in the frequency profile 11, also can be compared with frequencies of different body parts or body structures or types of tissue, stored in the database. Properties such as the type or composition or condition of the skin or tissue under examination, for example, can be deduced from the greatest similarity in the frequencies of maximum absorption or minimum reflection. This enables healthy tissue and diseased tissue to be distinguished and identified.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for examining a body or body structure, comprising:
detecting radiation emitted from and/or reflected by the body or body structure, said radiation being in the terahertz frequency range; and
evaluating the detected radiation to obtain information concerning the body or body structure.

2. The method according to claim 1, wherein detecting includes detecting radiation in the range between 0.1 THz and 30 THz.

3. The method according to claim 1, wherein evaluating includes determining information concerning at least one of a nature, type, composition, material, shape, structure, condition, temperature, surface location, or interior location of the body or body structure.

4. The method according to claim 3, further comprising registering the body or body structure based on the information concerning the body or body structure.

5. The method according to claim 1, further comprising combining the obtained information concerning the body or body structure with other information concerning the body or body structure.

6. The method according to claim 5, wherein combining includes performing an image fusion process between the obtained information concerning the body or body structure with the other information concerning the body or body structure.

7. The method according to claim 5, wherein the combined information forms data set.

8. The method according to claim 5, wherein combining includes using a two-dimensional and/or three-dimensional data set of the body or body structure as the other information concerning the body or body structure.

9. The method according to claim 8, wherein using the two-dimensional and/or three-dimensional data set includes using a data set ascertained via at least one of an x-ray method, a magnetic resonance method, a computer tomography method, an ultrasound method, a positron emission tomography (PET) method or a single photon emission computed tomography (SPECT) method,

10. The method according to claim 1, wherein detecting radiation emitted from and/or reflected by the body or body structure includes detecting radiation in the terahertz frequency range reflected on the body or body structure, transmitted through the body or body structure, or emitted by the body or body structure.

11. The method according to claim 10, further comprising detecting the terahertz radiation emitted, transmitted or reflected from the body or body structure when the body or body structure is covered by clothing.

12. The method according to claim 1, further comprising navigating an object based on the obtained information concerning the body or body structure.

13. The method according to claim 1, wherein evaluating the detected radiation to obtain information concerning the body or body structure includes determining from chronological information of the detected radiation a shape, structure, condition, position, or distance of the body or body structure or of a surface of the body or body structure.

14. The method according to claim 13, wherein determining from chronological information includes using transit time of the radiation as the chronological information.

15. The method according to claim 1, wherein evaluating the detected radiation includes ascertaining spectral information concerning the body or body structure, and comparing the ascertained spectral information with spectral information of known bodies or body structures so as to determine a nature, type, composition, material, shape, structure, condition, temperature or position of the surface or interior of the body or body structure.

16. The method according to claim 1, wherein evaluating the detected radiation includes ascertaining characteristic frequencies of the body or body structure, and comparing the ascertained characteristic frequencies with characteristic frequencies of known bodies or body structures to determine a nature, type, composition, material, shape, structure, condition, temperature or position of the surface or interior of the body or body structure.

17. The method according to claim 16, wherein the characteristic frequencies are resonant frequencies or absorption frequencies.

18. The method according to claim 1, wherein the body is a head, face, arm or hand.

19. The method according to claim 1, wherein the body structure is a patient's tissue, bones and/or bone structures, vessels, ligaments, tendons, teeth or skin.

20. The method according to claim 1, wherein detecting radiation includes detecting radiation in a frequency range of between 0.1 and 5 THz, between 0.1 and 0.6 THz, or between 0.5 and 2 THz.

21. The method according to claim 1, wherein detecting radiation includes detecting radiation having a frequency of about 1.6 THz, 2.5 THz or 3 THz.

22. A computer program embodied on a computer readable medium for examining a body or body structure, comprising:
code that directs the detection of radiation emitted from and/or reflected by the body or body structure, said radiation being in the terahertz frequency range; and
code that evaluates the detected radiation to obtain information concerning the body or body structure.

23. A device for examining a body, comprising:
a terahertz sensor for detecting terahertz radiation reflected from a body or body structure, transmitted through the body or body structure, or emitted by the body or body structure; and
a computational unit operatively coupled to the terahertz sensor, said computational unit operative to evaluate the detected terahertz radiation so as to determine information concerning the body or body structure.

24. The device according to claim 23, wherein the terahertz sensor is a terahertz camera.

25. The device according to claim 23, wherein the computational unit comprises a tracking system.

26. The device according to claim 23, further comprising a terahertz radiation source operative to emit terahertz radiation onto the body or body structure, wherein the terahertz radiation source is operatively coupled to the terahertz sensor and/or the computational unit.

27. The device according to claim 26, wherein the terahertz radiation source is at least one terahertz lamp.

28. The device according to claim 23, further comprising a database operatively coupled to the computational unit, said database including characteristic information on a plurality of bodies or body structures, wherein the computational unit is operative to compare the information concerning the body or body structure with the characteristic information on the plurality of bodies or body structures, and based on the comparison, determine a nature, type, composition, material, shape, structure, condition, temperature or position of the surface or interior of the body or body structure.

29. The device according to claim 23, further comprising:
a data output device for displaying the information concerning the body or body structure; and
a data input device for inputting characteristic information concerning the body or body structure into the database or for processing by the computational unit.

30. The device according to claim 29, wherein the input device is at least one of a keyboard, an x-ray device, a computer tomograph, a nuclear spin tomograph, an ultrasound tomograph, a positron emission tomograph or a SPECT tomograph.

31. The device according to claim 23, wherein the terahertz radiation source and/or terahertz sensor comprise a terahertz oscillator.
